(19) United States
(12) Patent Application Publication
(10) Pub. No.: US 2004/0247704 A1
(43) Pub. Date: Dec. 9, 2004

(54) Composition of alcoholic effects neutralization ingredients

(76) Inventors: Mao-Liang Chiang, Jungli City (TW); Li-Wei Chiang, Jungli City (TW); Li-Chin Chiang, Jungli City (TW)

Correspondence Address: TROXELL LAW OFFICE PLLC, SUITE 1404, 5205 LEESBURG PIKE, FALLS CHURCH, VA, 22041, US

(21) Appl. No.: 10/453,624

(22) Filed: Jun. 4, 2003

(51) Int. Cl. A61K 35/78; A61K 31/715

(52) U.S. Cl. 424/729; 424/725; 514/54

(57) ABSTRACT

A composition of alcoholic neutralization ingredients, essentially made of powder Atractylis ovata Thunb, Poria cocoa Wolf, Alisma plantago-aquatica L, Pachma hoelen Rumph, Glycyrrhiza glabra L., Cinnamomum loureiru Nees, Thea sinensis L., and Flos puerariae extracted from natural plants and saccharin to be fused in cold water for drink before or after the alcoholic drinks, so to alleviate the hangover by promoting metabolism of alcohol in one's livers to avoid getting drunk due to the alcohol accumulated inside one's body, and the functions of the kidneys and skins to facilitate urination and sweating to excrete the alcohol while keeping one calm down to stay away from violence, losing temper and dizziness usually observed on one who is drunk.

### BACKGROUND OF THE INVENTION

1. Field of the Invention

The present invention is related to a composition of ingredients having an alcohol neutralizing effect, and more particularly, to one made of natural plants to be fused in cold water for drink so to neutralize the effects of alcoholic drinks and alleviate the hangover to avoid making troubles due to getting drunk.

2. Description of the Prior Art

Since Taiwan is admitted to World Trade Organization (WTO), practically the control of imported liquors and tobaccos is lifted, and the justification of tariff has made the imported liquors more competitive, more and more adults under the strong sales campaigns by importers of liquors start to take more drinks at the proper time and occasion.

However, when one gets drunk, his heart rate is accelerating, his mind is not clear, and is prevented from making normal thinking and judgment, less capable of response, entering into a delirious state of mind, and making trouble in serious cases. Therefore, the government always proactively advises people not to take too much drink, not to drive after drinks so to avoid hurting themselves and innocent bystanders or damaging the social security.

### SUMMARY OF THE INVENTION

The primary purpose of the present invention is to provide a composition of alcoholic effects neutralization ingredients, essentially made of powder Atractylis ovata Thunb, Poria cocoa Wolf, Alisma plantago-aquatica L., Pachma hoelen Rumph, Glycyrrhiza glabra L., Cinnamomum loureiru Ness, Thea sinensis L., and Flos Puerariae extracted from natural plants and saccharin to be fuse in cold water for drink before or after the alcoholic drinks, so to alleviate the hangover by promoting metabolism of one's livers to avoid getting drunk due to the alcohol accumulated inside one's body, and the functions of the kidneys and skins to facilitate urination and sweating to excrete the alcohol while keeping one calm down to stay away from violence, touchy, and dizziness.

### DETAILED DESCRIPTION OF THE PREFERRED EMBODIMENTS

A composition of alcoholic effects neutralization of the present invention is comprised of Atractylis ovata Thunb, known for effects of dehumidifying, sweating, protecting one's stomach and spleen so to help digestion, facilitate urination, cure dropsy and promote sweating;

Poria cocoa Wolf, containing tuckahoe, glucose and oligosaccharide, giving tranquilizing effect, facilitating urination, curing vomit and insomnia due to heart palpitation;

Alisma plantago-aquatica L., known for facilitating urination, stopping vomit, preventing the stomach from accumulating too much water, quenching thirst, and making dizziness go away;

Pachma hoelen Rumph, for facilitating urination and neutralizing excessive body heat;

Thea sinensis L., beneficial for one's heart, facilitating urination, keeping one refreshed and improved memory due to containments of catechotannin caffeine, and Vitamin C;

Flos Puerariae, Adenine and Glutamic for disphoresis, antipyretic, protecting stomach and neutralizing alcoholic effect by driving alcoholic out of ones skin; and

Glycyrrhiza glabra L., Cinnamomum loureiru Nees, and saccharin are added to rid of the odor from Atractylis ovata Thunb, Poria cocoa Wolf, Pachma hoelen Rumph, and Flos Puerariae.

In preparation, those ingredients are ground into powders and then is mixed at the dosage of Atractylis ovata Thunb (4.5 g), Poria cocoa Wolf (4.5 g), Poria cocoa Wolf (4.5 g), Alisma plantago-aquatica L. (4.5 g), Pachma hoelen Rumph (4.5 g), Glycyrrhiza glabra L., (12 g), Cinnamomum loureiru Nees (3 g), Thea sinensis L. (2 g), Flos Puerariae (4G) and saccharin (0.2 g) to be fused in cold water for drink. If taken before having liquors, the composition promotes metabolism of alcohol by acting on one's livers, and facilitates urination and sweating, on one's kidneys and skin, thus to accelerate metabolism of alcoholic containment in one's body and to excrete the alcoholic containment through urination and sweating to avoid hangover. Meanwhile, the composition helps tranquilize one's mind and eliminate violence, losing temper and dizziness usually observed on one who is drunk.

Furthermore, the composition can be also taken after having liquors to alleviate vomit and soonest neutralize the alcoholic effects for one to stay calm and avoid making troubles, particularly, avoid driving for one's own good and public safety.

The composition disclosed in the present invention is made of natural plants and to be infused in water for drink to neutralize alcoholic effects. Therefore this application is duly filed accordingly. However, it should be noted that the preferred embodiment is given only for reference purpose and not to limit the present invention. Any other composition which characteristics that are resembling or similar to the present invention shall be deemed as falling within the objectives and the scope of claims to be claimed by the present invention.

1-3. (Canceled)

4. A composition having an alcohol neutralizing effect comprising:
a mixture having:
a) Atractylis ovata Thunb;
b) Poria cocoa Wolf including tuckahoe, glucose and oligosaccharide;
c) Alisma plantago-aquatica L;
d) Pachma hoelen Rumph;
e) Flos Puerariae, Adenine and Glutamic; and
f) Thea sinensis L. including caffeine, and Vitamin C grounded into powders, wherein the mixture is infused in cold water for drinking before and/or after having liquors, such that, when taken before having liquors, the composition promotes metabolism of alcohol by acting on a liver and facilitates urination from kidneys and sweating from skin to accelerate metabolism of alcoholic containment in a body and to excrete the alcoholic containment through urination and sweating to avoid a hangover, and the mixture provides a calming effect.

5. The composition according to claim 4, further comprising: Glycyrrhiza glabra L., Cinnamomum loureiru Nees, and saccharin added to the mixture to remove an odor from the Atractylis ovata Thunb, the Poria cocoa Wolf, the Pachma hoelen Rumph; and the Flos Puerariae.

6. The composition according to claim 5, wherein the mixture is mixed at the dosage of 4.5 g of Atractylis ovata Thunb, 4.5 g of Poria cocoa Wolf, 6 g of Alisma plantago-aquatica L., 4.5 g of Pachma hoelen Rumph, 12 g of Glycyrrhiza glabra L., 3 g of Cinnamomum loureiru Nees, 2 g of Thea sinensis L., 4 g of Flos Puerariae, and 0.2 g of saccharin.

7. The composition according to claim 6, further comprising an additional 4.5 g of Poria cocoa Wolf.
